(19) United States
(12) Patent Application Publication
(10) Pub. No.: US 2003/0065374 A1
(43) Pub. Date: Apr. 3, 2003

(54) Active fixation lead with helix extension indicator

(75) Inventor: Jordon D. Honeck, Maple Grove, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 09/968,656

(22) Filed: Oct. 1, 2001

(51) Int. Cl. A61N 1/05

(52) U.S. Cl. 607/127

(57) ABSTRACT

A medical electrical lead that includes an indicator member that acts as a visual aid in implanting an electrode into a body tissue while using a fluoroscope. In an example embodiment, a medical electrical lead assembly includes an electrode head member, an electrode and a shaft member, the shaft member being retractable and extendible within the electrode head member. Each of the electrode, electrode head member and shaft member have, respectively, a distal end and a proximal end, and the electrode head member has an outer surface. The medical lead assembly includes an indicator coil member disposed on the outer surface of the electrode head member that is spaced laterally away from the shaft member in a first retracted position. The shaft member moves in alignment with the indicator coil member in a second extended position to indicate that the electrode extends out from the electrode head member.

### FIELD OF THE INVENTION

The present invention generally relates to medical electrical leads and more specifically to medical leads utilizing extendible electrodes for anchoring the leads to the endocardial tissue.

### BACKGROUND OF THE INVENTION

Cardiac pacing leads electrically connect a cardiac pacemaker pulse generator to the heart tissue that is being stimulated. Pacing leads are not only used for pacing but are also used for sensing electrical signals produced by the heart. In the latter case, the electrical lead serves as a bi-directional pulse transmission link between the pacemaker and the heart. The electrical lead assembly includes a distal end having an electrode that contacts the endocardial tissue and a proximal end having a connector pin that receives a mating socket in the pacemaker. A flexible, coiled conductor surrounded by an insulating tube or sheath couples the connector pin at the proximal end and the electrode at the distal end.

To improve reliability, the electrical lead assembly is located and stabilized adjacent the stimulated or monitored tissue. One approach for affixing the electrical lead involves using a rotatable fixation helix, which exits the distal end of the lead assembly and penetrates the body tissue in a corkscrew fashion. The helix itself operates as an electrode or operates to anchor the electrode and lead assembly adjacent the body tissue. In one application, the fixation helix of the electrical lead assembly is coupled to a conductor extending through the lead assembly and rotates when rotating the conductor within the lead assembly. Improperly affixing the electrical lead to the body tissue results in mechanical instability, inaccurate sensing and decreased pacing efficiency. Mechanical instability of the electrical lead produces excessive trauma and inflammatory tissue reaction that increases the stimulation threshold of the heart or body tissue.

During insertion of the electrical lead into the heart, the physician may use a fluoroscope, a medical imaging device that generates an image on a display screen, to follow the progress. The physician may use the fluoroscope to determine the point at which the helix electrode penetrates the heart tissue. A disadvantage to anchoring the electrical lead with the helix electrode is the difficulty in verifying the degree of extension of the helix electrode relative to the lead assembly tip. The distal end portion of the lead assembly is not readily visible on the fluoroscope because of the insulative (e.g., polyurethane) tubular housing of the lead assembly. One approach to overcoming this disadvantage involves observing how far a tip of the helix electrode extends beyond the end of the lead assembly. However, fluoroscopically viewing the relative positions of the electrode tip and the lead assembly tip is difficult because of the lack of mass in the loosely spaced coils of the helix electrode.

There is a need for a medical electrical lead assembly that fluoroscopically gauges the precise location of the helix electrode extension with regard to the distal tip of the lead assembly, and that promotes adherence of the lead assembly to the organ tissue to reduce tissue damage and the associated inflammatory response.

A medical electrical lead assembly that addresses the aforementioned problems, as well as other related problems, is therefore desirable.

### SUMMARY OF THE INVENTION

Various embodiments of the present invention are directed to addressing the above and other needs for the purpose of visually determining with a medical imaging device, the amount that an electrode of a medical lead assembly extends beyond the lead assembly and the point at which it penetrates into a body tissue.

One embodiment of the invention is directed to an electrode head member enclosing an electrode and a shaft member, the shaft member being retractable and extendible within the electrode head member. Each of the electrode, electrode head member and shaft member have, respectively, a distal end and a proximal end, and the electrode head member has an outer surface. The electrode head member includes an indicator member disposed on the outer surface of the electrode head member and is spaced laterally away from the shaft member in a first retracted position. The shaft member moves in alignment with the indicator member in a second extended position to indicate that the electrode extends out from the electrode head member. In a related embodiment, the electrode head member is incorporated into a medical electrical lead assembly for use with an implanted medical device.

Another embodiment of the invention is directed to an electrode head member enclosing an electrode and a shaft member, the shaft member being retractable and extendible within the electrode head member. Each of the electrode, electrode head member and shaft member have, respectively, a distal end and a proximal end, and the electrode head member has an outer surface. The electrode head member includes complimentary C-shaped indicator members disposed on an outer surface of the electrode head member in a non-contacting ring arrangement. The C-shaped indicator members are spaced laterally away from the shaft member in a first retracted position. The shaft member moves in alignment with the C-shaped indicator members in a second extended position to indicate that the electrode extends out from the electrode head member.

The above summary of the present invention is not intended to describe each illustrated embodiment or every implementation of the present invention. The figures in the detailed description that follow more particularly exemplify these and other embodiments.

### BRIEF DESCRIPTION OF THE DRAWINGS

The invention may be more completely understood in consideration of the following detailed description of various embodiments of the invention in connection with the accompanying drawings, in which:

FIG. 1 illustrates a retracted electrode in a distal portion of an electrode head member of a lead assembly according to an example embodiment of the invention; and

FIG. 2 illustrates an extended electrode indicating arrangement of an electrode head member according to an example embodiment of the invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION

The present invention is generally directed to a visual aid located on a medical lead that is used with a medical imaging device to implant the medical lead into a body tissue of a patient. In a particular application, the visual aid provides feedback to a physician as to the degree that an electrode of the medical lead penetrates into the body tissue. While the present invention is not necessarily limited to such an application, the invention will be better appreciated using a discussion of example embodiments in such a specific context.

In an example embodiment, a medical electrical lead assembly includes a fixation helix electrode, an electrode head member and a shaft member, the shaft member being retractable and extendible within the electrode head member. Each of the electrode, electrode head member and shaft member have, respectively, a distal end and a proximal end, and the electrode head member has an outer surface. The medical lead assembly includes an indicator coil member disposed within a recessed portion of the outer surface of the electrode head member and is spaced laterally away from the shaft member in a first retracted position. The shaft member moves in alignment with the indicator coil member in a second extended position to indicate that the electrode extends out from the electrode head member. Aligning the indicator coil member with the distal end of the shaft member positively indicates that the electrode has penetrated the body tissue.

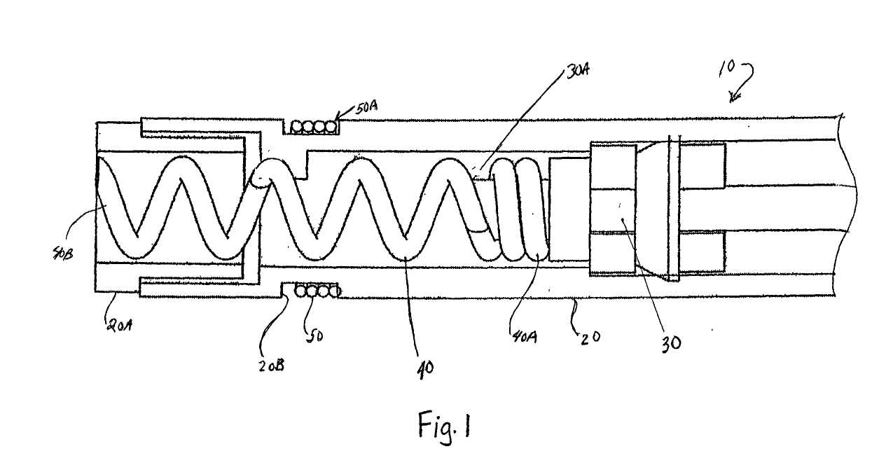

FIG. 1 illustrates a retracted electrode in a distal portion of an electrode head member of a lead assembly 10 according to an example embodiment of the invention. In this example embodiment, a portion of a cardiac pacing lead assembly 10 includes an electrode head member 20 that encapsulates an electrode 40 coupled to a shaft member 30. Electrode head member 20 is formed from a generally tubular molding of rigid, biocompatible plastic such as polyurethane, and includes a distal portion 20A that is molded from similar material of biocompatible silicone rubber, polyurethane or other biocompatible plastic. In a more specific example embodiment, distal portion 20A contains a steroid, such as a form of dexamethasone acetate, that elutes from the plastic after the lead assembly is affixed to the body tissue. The description of the present invention is directed primarily to the electrode head portion of a medical lead. For more information regarding the other components of a medical lead assembly, reference may be made to U.S. Pat. No. 5,948,015 to Hess et al., which is assigned to the assignee of the present invention and incorporated herein by reference.

In this example embodiment, electrode 40 is an advanceable fixation helix that is mechanically coupled to shaft member 30. Electrode 40 is shown in a retracted position. As shaft member 30 rotates clockwise relative to lead assembly 10, shaft member 30 starts to move forward toward a distal end 20A of electrode head member 20. The rotational movement of shaft member 30 is transferred to electrode 40 such that electrode 40 rotates and extends out from distal portion 20A. Electrode 40 retracts into electrode head member 20 as shaft member 30 rotates in a counterclockwise direction. For more information regarding advancement or retraction of the fixation helix within an electrode head member, reference may be made to U.S. Pat. No. 4,106,512 issued to Bisping et al., which is assigned to the assignee of the present invention and incorporated herein by reference.

During insertion of lead assembly 10 into the heart, the physician may use a fluoroscope to gauge the progress of penetration of electrode 40 into the heart tissue. Metallic components with dense structures, such as shaft member 30 or a tightly wound portion of electrode coil 40A, are visible under the fluoroscope. However, a loosely wound portion of electrode coil 40B may not be clearly visible to the physician because the spaced coils lack sufficient mass to be readily seen. To assist the physician, a radiopaque indicator coil member 50 is inserted into a recessed portion 20B of electrode head member 20. Indicator coil member 50 is spaced laterally away from shaft member 30 and toward distal portion 20A. When shaft member 30 is a retracted position, a gap exists between a distal end 30A of shaft member 30 and a proximal end 50A of coil member 50. The gap is visible to the physician under the fluoroscope. As the physician advances shaft member 30 to the extended position, electrode 40 advances and extends out from electrode head member 20. As shaft member 30 moves forward and aligns with coil member 50, the gap between shaft member 30 and indicator coil member 50 disappears.

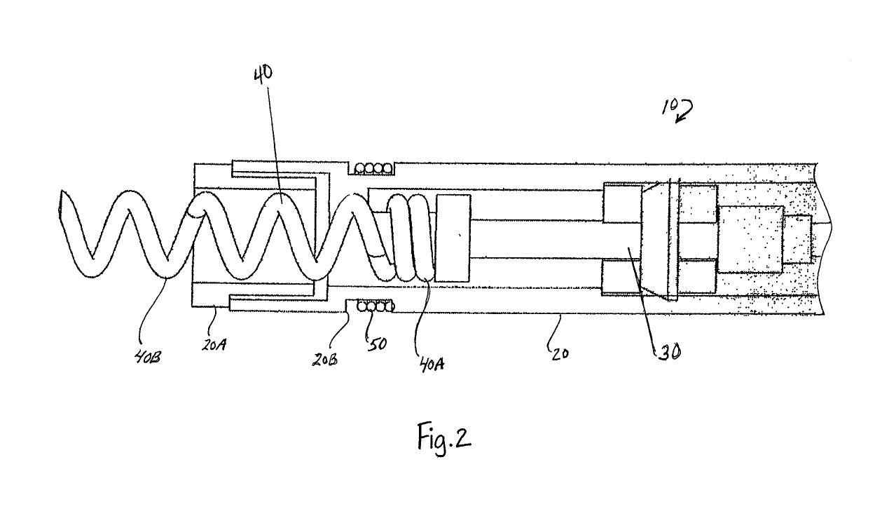

FIG. 2 illustrates electrode 40 fully extended or advanced from electrode head member 20. To extend the helix electrode, the physician turns shaft member 30 clockwise to advance the shaft member and the electrode, thereby aligning shaft member 30 with indicator coil member 50. The gap closes or disappears when the shaft member and coil member are substantially aligned. With the gap closed, the physician concludes that portion 40B of electrode 40 is fully extended. Electrode 40 is now fully extended although the loosely wound coil portion 40B of the electrode may not be clearly visible under the fluoroscope. The advancing rotational motion of electrode portion 40B as the electrode reaches full extension anchors the electrode to the body tissue. Gap closure positively indicates that the electrode is fully extended and that it is safe to stop turning the shaft member any further. The amount that electrode 40 extends from distal portion 20 A is inversely proportional to the gap size. That is, the length that electrode portion 40B extends from distal portion 20A increases as the gap size decreases.

With the present invention, lead assembly 10 can be further secured to the body tissue with indicator coil member 50, because coil member 50 is made of wire material having a rough surface that enhances the frictional adherence of the lead assembly to the body tissue. In contrast, if coil member 50 were made of a smooth wire material, the uneven surface of the coil member would also increase adherence to the body tissue because the blood would pool in the “hills and valleys” of the coil surface and later form into cell adhesion.

In addition to easily verifying electrode insertion, another application of the present invention simplifies the process of removing electrode portion 40B from the body tissue because the physician has a positive visible indicator that electrode 40 is fully retracted. Under the fluoroscope, as electrode 40 is retracted, the gap between coil member 50 and shaft member is again established. The presence of the gap positively indicates that the electrode is fully retracted, and the lead assembly can be withdrawn. In one example embodiment, indicator coil member 50 is comprised of tightly wound coils of a platinum-iridium alloy or tantalum wire having a thickness of about 1-5 mils (thousandths of an inch). In another related embodiment, coil member 50 is comprised of a tantalum wire that may include an insulative oxide coating for substantially reducing induction current effects between the electrode and another lead on lead assembly 10. The diameter of the coils of coil member 50 is sized to be about one-half the diameter of the electrode head member 50 to form a snug fit within recessed portion 20B. Coil member 50 can also be further secured to the electrode head member 50 with a biocompatible adhesive.

In another embodiment, recessed portion 20B is substitutable with one or more threads that engage indicator coil member 50 and secure the coil member to the electrode head member 20. Indicator coil member 50 can also be located on other areas of electrode head member 20, such as the midsection, and is not necessarily limited to the location shown in FIGS. 1 and 2.

In another embodiment, the indicator member is comprised of two complementary C-shaped radiopaque members that are disposed on the top and bottom of the electrode head member in a non-contacting ring arrangement. The C-shaped members can be attached to the electrode head member with a biocompatible adhesive or can be pressure fit into the recessed portion 20B of the electrode head member.

Accordingly, various aspects of the present invention provide the advantage of not increasing the electrode head member size when using a monolithic controlled release device (MCRD). These aspects of the invention are useful in implantable cardioversion and defibrillation leads and in other leads that benefit from affixing the lead to the body tissue to stimulate or sense electrical activity in the body, including nerve and muscle stimulation leads.

Various modifications, equivalent processes, as well as numerous structures to which the present invention may be applicable will be readily apparent to those of skill in the art to which the present invention is directed upon review of the present specification. The claims are intended to cover such modifications and devices.

1. An electrode head member enclosing an electrode and a shaft member, the shaft member being retractable and extendible within the electrode head member, each of the electrode, electrode head member and shaft member having, respectively, a distal end and a proximal end, the electrode head member having an outer surface, the electrode head member comprising:
an indicator member disposed on the outer surface of the electrode head member, the indicator member spaced apart from the shaft member in a first retracted position, the shaft member adapted to move in alignment with the indicator member in a second extended position thereby indicating that the electrode extends out from the electrode head member.

2. The electrode head member of claim 1, wherein the electrode includes a fixation helix electrode.

3. The electrode head member of claim 2, wherein helix electrode further comprises a proximal portion with closely wound coils and a distal portion with spaced coils.

4. The electrode head member of claim 1, wherein the indicator member is a coiled member spaced laterally from the distal end of the shaft member.

5. The electrode head member of claim 1, wherein the indicator member is disposed within a recessed portion of the electrode head member.

6. The electrode head member of claim 5, wherein the recessed portion is disposed adjacent the distal end of the electrode head member.

7. The electrode head member of claim 4, wherein the indicator member is in contact with a raised thread member of the electrode head member adapted to engage at least one coil of the coiled member.

8. The electrode head member of claim 1, wherein the indicator member is configured with a rough surface to increase frictional adherence of the lead assembly to a body tissue.

9. The electrode head member of claim 1, wherein the indicator member is comprised of a tantalum wire coated with an oxide layer, thereby reducing induction current effects.

10. The electrode head member of claim 1, wherein the indicator member is comprised of a wire made of a material selected from the group consisting of: tantalum, platinum and iridium.

11. A medical electrical lead assembly having an electrode head member, an electrode and a shaft member, the shaft member being retractable and extendible within the electrode head member, each of the electrode, electrode head member and shaft member having, respectively, a distal end and a proximal end, the electrode head member having an outer surface, the medical electrical lead assembly comprising:
an indicator coil member disposed on the outer surface of the electrode head member, the indicator coil member spaced laterally away from the shaft member in a first retracted position, the shaft member adapted to move in alignment with the indicator coil member in a second extended position thereby indicating that the electrode extends out from the electrode head member.

12. The medical lead assembly of claim 11, wherein the indicator coil member is disposed within a recessed portion of the distal end of the electrode head member.

13. The medical lead assembly of claim 12, wherein the indicator coil member is configured with a rough surface to increase adherence of the lead assembly to a body tissue.

14. The medical lead assembly of claim 13, wherein the electrode head member further comprises a monolithic controlled release device for eluting an anti-inflammatory agent into the body tissue.

15. The medical lead assembly of claim 11, wherein the electrode includes a fixation helix electrode.

16. The medical lead assembly of claim 11, wherein the indicator coil member is comprised of a tantalum wire coated with an oxide layer, thereby eliminating induction current effects.

17. The medical lead assembly of claim 11, wherein the indicator coil member is comprised of a wire made of a material selected from the group consisting of: tantalum, platinum and iridium.

18. The medical lead assembly of claim 11, wherein the indicator coil member is in contact with a raised thread member of the electrode head member adapted to engage at least one coil of the indicator coil member.

19. The medical lead assembly of claim 12, wherein the indicator coil member is sized with a diameter of about half of a diameter of the electrode head member, thereby reducing coil member movement within the recessed portion.

20. An electrode head member enclosing an electrode and a shaft member, the shaft member being retractable and extendible within the electrode head member, each of the electrode, electrode head member and shaft member having, respectively, a distal end and a proximal end, the electrode head member having an outer surface, the electrode head member comprising:
one or more indicator members disposed on an outer surface of the electrode head member in a non-contacting ring arrangement, the one or more indicator members being spaced laterally away from the shaft member in a first retracted position, the shaft member adapted to move in alignment with the one or more indicator members in a second extended position thereby indicating that the electrode extends out from the electrode head member.

21. The electrode head member of claim 21, wherein the one or more indicator members comprise multiple complementary C-shaped indicator members.
